(19) United States
(12) Patent Application Publication
(10) Pub. No.: US 2023/0355608 A1
(43) Pub. Date: Nov. 9, 2023

(54) TARGETING POLYCYTHEMIA VERA (PV)-INITIATING CELLS BY BLOCKING LEUKOTRIENE BINDING TO THEIR RECEPTORS

(71) Applicant: UNIVERSITY OF MASSACHUSETTS, Boston, MA (US)

(72) Inventor: Shaoguang Li, Worcester, MA (US)

(21) Appl. No.: 18/028,991

(22) PCT Filed: Sep. 24, 2021
(86) PCT No.: PCT/US2021/051975

§ 371 Date: Mar. 28, 2023

Related U.S. Application Data
(60) Provisional application No. 63/086,936, filed on Oct. 2, 2020.

(51) Int. Cl. A61K 31/47; A61K 31/519; A61K 31/5377; A61K 31/506; A61K 31/553; A61P 35/00; A61K 35/28; C12N 5/0789

(52) U.S. Cl. CPC A61K 31/47; A61K 31/519; A61K 31/5377; A61K 31/506; A61K 31/553; A61P 35/00; A61K 35/28; C12N 5/0647; C12N 2501/727; C12N 2501/998; C12N 2501/2303

(57) ABSTRACT

This invention relates to treating Myeloproliferative neoplasms (MPNs), specifically Polycythemia Vera (PV). In particular, the invention relates to compositions and methods for inhibiting Polycythemia Vera (PV)-initiating cells, e.g. subsets of hematopoietic stem cells that cause PV symptoms. For one example, Montelukast (e.g., Singulair®) may be used for preventing the development of PV or reducing PV symptoms over long periods of time. Further, Montelukast in combination with other compounds, e.g., JAK2 inhibitor compounds, may also find use for treating patients genetically at risk for developing PV and for PV patients.

### STATEMENT OF GOVERNMENT SUPPORT

This invention was made with government support under grant number CA176179 awarded by the National Institutes of Health. The government has certain rights in the invention.

### FIELD OF THE INVENTION

This invention relates to treating Myeloproliferative neoplasms (MPNs), specifically Polycythemia Vera (PV). In particular, the invention relates to compositions and methods for inhibiting Polycythemia Vera (PV)-initiating cells, e.g. subsets of hematopoietic stem cells that cause PV symptoms. For one example, Montelukast (e.g., Singulair®) may be used for preventing the development of PV or reducing PV symptoms over long periods of time. Further, Montelukast in combination with other compounds, e.g., JAK2 inhibitor compounds, may also find use for treating patients genetically at risk for developing PV and for PV patients.

### BACKGROUND

Myeloproliferative neoplasms (MPNs), such as polycythemia vera (PV), essential thrombocythemia (ET), myelofibrosis (MF) and idiopathic myelofibrosis (IMF), are hematologic malignancies with a chronic clinical course having a risk of further developing thrombosis, acute leukemia. MPNs arise from aberrant cells in the hematopoietic stem cell (HSC) compartment of differentiating/maturing cells having certain genetic mutations. One frequently present mutant allele in humans that is directly related to MPNS is allele JAK2V617F. JAK2 gene alleles were sequenced as a member of a family of Janus kinases, including JAK1, JAK2 and JAK3 and tyrosine kinase 2. The JAK2V617F allele contains a genetic point mutation resulting in substituting an allele encoding valine that instead codes for phenylalanine in the JAK2 protein.

JAK2V617F alleles were found in MPN patient populations, e.g., up to 100% of PV patients, 40% to 70% of ET patients, and approximately 50% of patients with acute myeloid leukemia (AML). Further, allele JAK2V617F was found in a small subset of the hematopoietic population, suggesting these are PV-initiating cells. Moreover, hematopoietic stem cells from MPN patients in general are hypersensitive to a range of growth factors, including those associated with JAK2 for transmembrane signaling in cells.

Thus, current therapy of MPN patients mainly focuses on inhibition of mutant (overactive) JAK2 V617F kinase activity using JAK2 inhibitor compounds; however they do not inhibit effects of MPN-initiating cells prior to maturation. Further, current therapies are not desirable to use for multiple reasons including a lack of effectiveness in reducing PV symptoms while inducing toxicity symptoms over multiple treatments. Moreover some patients become resistant to benefits of therapeutic compounds because once a patient develops PV, it must be treated for the remainder of the patient's life.

Therefore, an effective therapeutic strategy that aims to reduce symptoms without toxic side effects is needed. Further, an effective therapeutic strategy in patients at risk of developing PV for inhibiting the development of PV symptoms is also needed.

### SUMMARY OF THE INVENTION

This invention relates to treating Myeloproliferative neoplasms (MPNs), specifically Polycythemia Vera (PV). In particular, the invention relates to compositions and methods for inhibiting Polycythemia Vera (PV)-initiating cells, e.g. subsets of hematopoietic stem cells that cause PV symptoms. For one example, Montelukast (e.g., Singulair®) may be used for preventing the development of PV or reducing PV symptoms over long periods of time. Further, Montelukast in combination with other compounds, e.g., JAK2 inhibitor compounds, may also find use for treating patients genetically at risk for developing PV and for PV patients.

In one embodiment, the present invention contemplates a method of treatment, comprising: a) providing; i) a patient having Polycythemia Vera or is at risk of having Polycythemia Vera; and ii) a therapeutic composition comprising an active component consisting essentially of Montelukast; and b) administering said therapeutic composition to said patient wherein development of said Polycythemia Vera is inhibited development in said patient. In one embodiment, the Montelukast is selected from the group consisting of Montelukast sodium; Montelukast nitrile; and [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid, monosodium salt).

In one embodiment, the patient comprises hematopoietic stem cells (HSCs) having a JAK2V617F mutation. In one embodiment, the method further comprises isolating a first sample comprising a first plurality of hematopoietic stem cells from said patient, wherein a percentage of said first plurality of hematopoietic stem cells have over-active Janus kinase 2 (JAK2). In one embodiment, the method further comprises isolating a second sample comprising a second plurality of hematopoietic stem cells from said patient, wherein a lower percentage of said second plurality of hematopoietic stem cells have over-active Janus kinase 2 (JAK2) as compared to said first plurality of hematopoietic stem cells. In one embodiment, the first plurality of hematopoietic stem cells further comprise a percentage hematopoietic stem cells that are over-expressing arachidonate 5-lipoxygenase (Alox5). In one embodiment, the second plurality of hematopoietic stem cells further comprise a lower percentage of said hematopoietic stem cells that are overexpressing arachidonate 5-lipoxygenase (Alox5) than said first plurality of hematopoietic stem cells. In one embodiment, the patient is human.

In one embodiment, the present invention contemplates a method of treatment, comprising: a) providing; i) a patient having, or is at risk of having, Polycythemia Vera; and ii) a therapeutic composition comprising an active component consisting essentially of a combination of montelukast sodium and a JAK2 inhibitor compound; and b) administering said therapeutic composition to said patient wherein development of said Polycythemia Vera is inhibited. In one embodiment, the JAK2 inhibitor compound is ruxolitinib. In one embodiment, the JAK2 inhibitor compound is selected from the group consisting of redratinib, SAR317461, gandotinib, CEP-701

In one embodiment, the present invention contemplates a method of treatment, comprising: a) providing; i) a patient having, or is at risk of having, Polycythemia Vera; and ii) a therapeutic composition comprising an active component consisting essentially of a combination of montelukast sodium and a tyrosine kinase inhibitor compound; and b) administering said therapeutic composition to said patient wherein the development of said Polycythemia Vera is inhibited. In one embodiment, the tyrosine kinase inhibitor compound is selected from the group consisting of a Bcr-Abl tyrosine kinase inhibitor compound, AMN107; and AEE788.

In one embodiment, the present invention contemplates a method of treatment, comprising: a) providing; i) a population of CD34+ cells isolated from a patient having, or is at risk of having, Polycythemia Vera; and ii) a therapeutic composition comprising an active components consisting essentially of a combination of Montelukast and a kinase inhibitor compound; and b) culturing said CD34+ cells wherein a growing population of said cells is created; and c) administering said therapeutic composition to said growing population of CD34+ cells, such that cell growth of said growing population of CD34+ is reduced. In one embodiment, the kinase inhibitor compound is a JAK2 inhibitor compound. In one embodiment, the kinase inhibitor compound is a tyrosine kinase inhibitor compound. In one embodiment, the method further comprises isolating a first sample from said growing population of CD34+ cells, such that a percentage of said CD34+ cells have over-active Janus kinase 2 (JAK2). In one embodiment, the method further comprises isolating a second sample from said growing population CD34+ cells, wherein a lower percentage of said CD34+ cells have over-active Janus kinase 2 (JAK2) than said first sample. In one embodiment, the first sample further comprises a percentage of CD34+ cells that are over-expressing arachidonate 5-lipoxygenase (Alox5). In one embodiment, the second sample further comprises a lower percentage of said CD34+ cells overexpressing arachidonate 5-lipoxygenase (Alox5) than said first sample. In one embodiment, the method further comprises treating said patient with said reduced growth CD34+ cell population wherein development of, or a symptom of, Polycythemia Vera is reduced.

In one embodiment, the present invention contemplates a method of treatment, comprising, a) providing, i) a patient having Polycythemia Vera or at risk of having Polycythemia Vera; and ii) a therapeutic composition whose active component consists of Montelukast; and b) administering said therapeutic composition to said patient for inhibiting PV-initiating cells and inhibiting PV development in said patient. In one embodiment, said Montelukast is Montelukast sodium. However it is not intended to limit the type of Montelukast such that Montelukast may be Montelukast nitrile and [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid, monosodium salt). In one embodiment, a sample comprising hematopoietic stem cells is isolated from said patient, wherein nonlimiting examples of isolation include but are not limited to a blood sample comprising white blood cells, a bone marrow sample, a biopsy sample, a bone marrow biopsy sample, a lymph node sample, etc. In one embodiment, said sample comprising hematopoietic stem cells is isolated from said patient, wherein said hematopoietic stem cells have a JAK2V617F mutation. In one embodiment, wherein a sample comprising hematopoietic stem cells is isolated from said patient before step b), such that a percentage of said hematopoietic stem cells have over-activate Janus kinase 2 (JAK2). In one embodiment, wherein after step b) a sample comprising hematopoietic stem cells is isolated from said patient, such that a lower percentage of said hematopoietic stem cells have over-activate Janus kinase 2 (JAK2) than cells isolated before step b). In one embodiment, wherein a sample comprising hematopoietic stem cells is isolated from said patient before step b), wherein a percentage of hematopoietic stem cells are over-expressing arachidonate 5-lipoxygenase (Alox5). In one embodiment, wherein after step b) a sample comprising hematopoietic stem cells is isolated from said patient, such that a lower percentage of said hematopoietic stem cells are overexpressing arachidonate 5-lipoxygenase (Alox5) than cells isolated before step b). In one embodiment, said patient is human.

In one embodiment, the invention provides a method of treatment, comprising, a) providing, i) a patient having Polycythemia Vera; and ii) a therapeutic composition whose active component consists of Montelukast; and b) administering said therapeutic composition to said patient for inhibiting PV-initiating cells.

In one embodiment, the invention provides a method of treatment, comprising, a) providing, i) a patient having Polycythemia Vera; and ii) a therapeutic composition whose active component consists of Montelukast; and b) administering said therapeutic composition to said patient for inhibiting PV development in said patient.

In one embodiment, the invention provides a method of treatment, comprising, a) providing, i) a patient at risk of having Polycythemia Vera; and ii) a therapeutic composition whose active component consists of Montelukast; and b) administering said therapeutic composition to said patient for inhibiting PV-initiating cells.

In one embodiment, the invention provides a method of treatment, comprising, a) providing, i) a patient at risk of having Polycythemia Vera; and ii) a therapeutic composition whose active component consists of Montelukast; and b) administering said therapeutic composition to said patient for inhibiting PV development in said patient.

In one embodiment, the invention provides a method of treatment, comprising, a) providing, i) a patient having or at risk of having Polycythemia Vera; and ii) a test therapeutic composition whose active component consists of an antagonist for a Cysteinyl leukotriene receptor 1; and b) administering said therapeutic composition to said patient for inhibiting PV-initiating cells and inhibiting PV development in said patient. In one embodiment, said Cysteinyl leukotriene receptor 1 antagonist is zafirlukast. In one embodiment, said Cysteinyl leukotriene receptor 1 antagonist is pranlukast. In one embodiment, said Cysteinyl leukotriene receptor 1 antagonist is a test compound.

In one embodiment, the invention provides a method of treatment, comprising, a) providing, i) a patient having or at risk of having Polycythemia Vera; and ii) a test therapeutic composition whose active component comprises an antagonist for a Cysteinyl leukotriene receptor 1; and b) administering said therapeutic composition to said patient for inhibiting PV-initiating cells and inhibiting PV development in said patient. In one embodiment, said Cysteinyl leukotriene receptor 1 antagonist is zafirlukast. In one embodiment, said Cysteinyl leukotriene receptor 1 antagonist is pranlukast. In one embodiment, said Cysteinyl leukotriene receptor 1 antagonist is a test compound. In one embodiment, said method further comprises, a test kinase inhibitor compound and a step before c), administering said test kinase inhibitor compound. In one embodiment, said method further comprises a test kinase inhibitor compound and a step d) after step c), administering said test kinase inhibitor compound. In one embodiment, said test kinase inhibitor compound is a Janus kinase 2 inhibitor compound. In one embodiment, said Janus kinase 2 inhibitor compound is ruxolitinib. In one embodiment, said JAK2 inhibitor compound is selected from the group consisting of redratinib, SAR317461, gandotinib, CEP-701, etc. In one embodiment, said test kinase inhibitor compound is a tyrosine kinase inhibitor compound. In one embodiment, said test kinase inhibitor compound is selected from the group consisting of a Bcr-Abl tyrosine kinase inhibitor compound, e.g., imatinib; AMN107; AEE788, a multiple target tyrosine kinase inhibitor compound for reducing phosphorylation of the tyrosine kinases of epidermal growth factor receptor (EGFR), human epidermal growth factor receptor 2 (HER2), and vascular endothelial growth factor receptor 2 (VEGF2), etc.

In one embodiment, the invention provides a method of treatment, comprising, a) providing, i) a patient having or at risk of having Polycythemia Vera; and ii) a test therapeutic composition whose active component consists of an antagonist for a Cysteinyl leukotriene receptor 1 and a Janus kinase 2 inhibitor compound; and b) administering said therapeutic composition to said patient for inhibiting PV-initiating cells and inhibiting PV development in said patient. In one embodiment, said Cysteinyl leukotriene receptor 1 antagonist is zafirlukast. In one embodiment, said Cysteinyl leukotriene receptor 1 antagonist is pranlukast. In one embodiment, said Cysteinyl leukotriene receptor 1 antagonist is a test compound.

In one embodiment, the invention provides a method of treatment, comprising, a) providing, i) a patient having or at risk of having Polycythemia Vera; and ii) a test therapeutic composition whose active component consists of an antagonist for a Cysteinyl leukotriene receptor 1 and a kinase inhibitor compound; and b) administering said therapeutic composition to said patient for inhibiting PV-initiating cells and inhibiting PV development in said patient. In one embodiment, said Cysteinyl leukotriene receptor 1 antagonist is zafirlukast. In one embodiment, said Cysteinyl leukotriene receptor 1 antagonist is pranlukast. In one embodiment, said Cysteinyl leukotriene receptor 1 antagonist is a test compound.

In one embodiment, the invention provides a method of treatment, comprising, a) providing, i) a patient having or at risk of having Polycythemia Vera; and ii) a test therapeutic composition whose active component consists of Zileuton and a Janus kinase 2 inhibitor compound; and b) administering said therapeutic composition to said patient for inhibiting PV-initiating cells and inhibiting PV development in said patient.

In one embodiment, the invention provides a method of treatment, comprising, a) providing, i) a patient having or at risk of having Polycythemia Vera; and ii) a test therapeutic composition whose active component consists of Zileuton and a tyrosine kinase inhibitor compound; and b) administering said therapeutic composition to said patient for inhibiting PV-initiating cells and inhibiting PV development in said patient.

In one embodiment, the invention provides a method of treatment, comprising, a) providing, i) a patient having or at risk of having Polycythemia Vera; and ii) a test therapeutic composition whose active component consists of a leukotriene synthesis inhibitor compound; and b) administering said therapeutic composition to said patient for inhibiting PV-initiating cells and inhibiting PV development in said patient. In one embodiment, said leukotriene synthesis inhibitor compound is zileuton. In one embodiment, said leukotriene synthesis inhibitor compound is Hypericum perforatum. In one embodiment, said leukotriene synthesis inhibitor compound is a test compound. In one embodiment, said method further comprises, a test kinase inhibitor compound and a step before c), administering said test kinase inhibitor compound. In one embodiment, said method further comprises a test kinase inhibitor compound and a step d) after step c), administering said test kinase inhibitor compound. In one embodiment, said test kinase inhibitor compound is a Janus kinase 2 inhibitor compound. In one embodiment, said JAK2 inhibitor compound is ruxolitinib. In one embodiment, said JAK2 inhibitor compound is selected from the group consisting of redratinib, SAR317461, gandotinib, CEP-701, etc. In one embodiment, said test kinase inhibitor compound is a tyrosine kinase inhibitor compound. In one embodiment, said test kinase inhibitor compound is selected from the group consisting of a Bcr-Abl tyrosine kinase inhibitor compound, e.g., imatinib; AMN107; AEE788, a multiple target tyrosine kinase inhibitor compound for reducing phosphorylation of the tyrosine kinases of epidermal growth factor receptor (EGFR), human epidermal growth factor receptor 2 (HER2), and vascular endothelial growth factor receptor 2 (VEGF2), etc. In one embodiment, said test kinase inhibitor compound is not an imidazopyridine compound.

In one embodiment, the invention provides a method of treatment, comprising, a) providing, i) a population of CD34+ cells isolated from a patient having or at risk of having Polycythemia Vera; and ii) a therapeutic composition whose active component consists of Montelukast; and b) culturing said CD34+ cells under conditions for providing a growing population of said cells; and c) administering said therapeutic composition to said growing CD34+ cells, such that cell growth is reduced. In one embodiment, said population of CD34+ cells comprises a percentage of cells having a JAK2V617F mutation, wherein after step c), said percentage of JAK2V617F positive cells is lower than a duplicate population of cells growing without step c).

In one embodiment, the invention provides a method of treatment, comprising, a) providing, i) a population of CD34+ cells isolated from a patient having or at risk of having Polycythemia Vera; and ii) a therapeutic composition whose active component consists of Montelukast and a Janus kinase 2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of Montelukast and a test Janus kinase 2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of Montelukast and a tyrosine kinase inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera (PV); and ii) a therapeutic composition whose active components consist of Montelukast and a kinase inhibitor compound; and b) culturing said CD34+ cells under conditions for providing a growing population of said cells; and c) administering said therapeutic composition to said growing CD34+ cells, such that cell growth is reduced. In one embodiment, said kinase inhibitor compound is a JAK2 inhibitor compound. In one embodiment, said kinase inhibitor compound is a tyrosine kinase inhibitor compound. In one embodiment, wherein a sample is isolated from said population of cells before step b), such that a percentage of said hematopoietic stem cells have over-activate Janus kinase 2 (JAK2). In one embodiment, wherein after step c) a cell sample is isolated from said culture, wherein a lower percentage of said hematopoietic stem cells have over-activate Janus kinase 2 (JAK2) than said percentage of cells isolated before step b). In one embodiment, wherein a sample comprising hematopoietic stem cells is isolated from said patient before step b), wherein a percentage of hematopoietic stem cells are over-expressing arachidonate 5-lipoxygenase (Alox5). In one embodiment, after step c) a cell sample is isolated from said culture, wherein a lower percentage of said hematopoietic stem cells overexpressing arachidonate 5-lipoxygenase (Alox5) than said percentage of cells isolated before step b). In one embodiment, wherein after reduction of cell growth, said therapeutic composition is used for treating a patient at risk of developing PV for delaying onset of at least one PV symptom or used for treating a patient having PV for reducing at least one symptom of PV. In one embodiment, wherein after reduction of cell growth, said therapeutic composition is used for treating a patient for reducing CD34+ cells involved with causing PV. In one embodiment, wherein after reduction of cell growth, said therapeutic composition is used for treating a patient at risk of developing PV for delaying onset of at least one PV symptom. In one embodiment, wherein after reduction of cell growth, said therapeutic composition is used for treating a patient having PV for reducing severity of at least one PV symptom.

In one embodiment, the invention provides a method of treatment, comprising, a) providing, i) a population of CD34+ cells isolated from a patient having or at risk of having Polycythemia Vera; and ii) a therapeutic composition whose active component consists of Montelukast and a test kinase inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of a test antagonist for a Cysteinyl leukotriene receptor 1; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of a test antagonist for a Cysteinyl leukotriene receptor 1 and a test Janus kinase 2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of a test antagonist for a Cysteinyl leukotriene receptor 1 and a test kinase inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises Zileuton and a JAK2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises Zileuton and a Janus kinase 2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises Zileuton and a tyrosine kinase inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component consists of a test leukotriene synthesis inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises a test leukotriene synthesis inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises a test leukotriene synthesis inhibitor compound; and b) culturing said CD34+ cells under conditions for providing a growing population of said cells; and c. administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises a test leukotriene synthesis inhibitor compound and a Janus kinase 2 inhibitor compound; and b) culturing said CD34+ cells under conditions for providing a growing population of said cells; and c) administering said therapeutic composition to said growing CD34+ cells, such that cell growth is reduced.

In one embodiment, the invention provides a method of treatment, comprising, a) providing, i) a population of CD34+ cells isolated from a patient having or at risk of having Polycythemia Vera; and ii) a therapeutic composition whose active component comprises a test leukotriene synthesis inhibitor compound and a tyrosine kinase inhibitor compound; and b) culturing said CD34+ cells under conditions for providing a growing population of said cells; and c) administering said therapeutic composition to said growing CD34+ cells, such that cell growth is reduced.

In some embodiments, methods of treatments described herein are contemplated for use in cell culture screening of test compounds for use in treating patients.

In one embodiment, the invention provides a method of treatment, comprising, a) providing, i) a population of CD34+ cells isolated from a patient having or at risk of having a myeloproliferative neoplasm; and ii) a therapeutic composition whose active component comprises a test leukotriene synthesis inhibitor compound; and b) culturing said CD34+ cells under conditions for providing a growing population of said cells; and c) administering said therapeutic composition to said growing CD34+ cells, such that cell growth is reduced.

### BRIEF DESCRIPTION OF THE DRAWINGS

FIGS. 1A-1I show exemplary results of Montelukast treatment on several cell types in vitro, including mouse cells and human cells.

FIGS. 1A, 1B and 1C shows there is dose-dependent growth inhibition with Montelukast treatment at 24 and 48 hour post-treatment time points on these exemplary cell types.

FIG. 1D shows that Montelukast had no significant cell-cycle block effect on parental mouse Ba/F3 cells that do not express JAK2V617F.

FIGS. 1E and 1F shows that Montelukast treatment caused a blockade of cell-cycle progression from G₀ to G₁ phase and G₁ to S+G₂−M phase in both mouse Ba/F3-JAK2V617F and human HEL cells.

FIGS. 1G, 1H and 1I shows that Montelukast treatment induced more dramatic apoptosis in mouse Ba/F3-JAK2V617F cells and human HEL cells compared with mouse Ba/F3 cells.

FIGS. 2A-2I show exemplary results of Montelukast treatment on several cell types over time and immunostained micrographs showing changes in cell morphology of treated cells.

FIGS. 2A and 2B show results of peripheral blood (PB) cells of PV mice treated with Montelukast. Both GFP+Gr−1+PV cell number and percentage increased more slowly with the treatment of Montelukast compared with placebo-treated group from 3rd month post treatment.

FIG. 2C shows that GFP−Gr−1+ cell number did not show a significant difference between two treatment groups: placebo vs. Montelukast.

FIG. 2D shows the effect of Montelukast on PV development consistent with PB (peripheral blood) cells as evidenced by less PV cell infiltration to the spleens compared to the placebo-treated group.

FIGS. 2E and 2F shows that Montelukast did not significantly affect the level of RBC (red blood cells) and Platelet Thrombocyte count (PLT) but had an inhibitor effect on Hemoglobin (HGB) and hematocrit (HCT) (FIGS. 2G and 2H).

FIGS. 2G and 2H show that Montelukast did not significantly affect the level of RBC and PLT values.

FIG. 2I shows that Montelukast treatment efficiently relieved fibrosis morphology in bone marrow of PV mice.

FIGS. 3E-3F show exemplary results of Montelukast treatment using flow cytometric (FCM) analysis.

FIGS. 3A and 3B show that flow cytometric (FCM) analysis of Montelukast treatment caused a dramatic decrease in both GFP+PV cell number and percentage in bone marrow (BM) cells.

FIGS. 3C, 3D, 3G, and 3H show that neither GFP−(minus) BM or GFP-Lin-c-Kit+Sca−1+ demonstrates a noteworthy difference between Montelukast-treated group and placebo-treated group.

FIGS. 3E and 3F show that both the percentage and number of PV stem cells (PSCs), as GFP+Lin−c−Kit+Sca−1+, dropped to a much lower level with the treatment of Montelukast compared to placebo-treated groups.

FIG. 4 shows exemplary results of in vitro treatment of wild-type (WT) murine stem cells with Zileuton; wild-type (WT) murine stem cells and Alox5 mutant murine stem cells expressing green fluorescent protein (GFP) Singulair®, Leukotriene and combinations of Singulair® (S) at 2 different concentrations (10 uM and 30 uM) with Leukotriene (LT). DMSO was used for a control.

FIG. 5 shows exemplary results of in vivo treatment of PV mice, with Singulair® vs a placebo, after infusion with GFP expressing cells, demonstrating a loss of the GFP+ cells (by 6 and 10 weeks post treatment) while non GFP (GFP−) cells showed a loss at 10 weeks. Singulair® caused a dramatic decrease in both GFP+PV cell number and percentage in BM, after 4 months treatment.

FIG. 6 shows exemplary results on specific cell types, after in vivo treatment of PV mice, with Singulair® vs a placebo, observed up to 150 days.

FIG. 7 shows exemplary results on specific cell types, after in vivo treatment of PV mice, with Singulair® vs a placebo, observed over a 5 month treatment period.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, “Polycythemia vera” or “PV” refers to a hematopoietic stem cell disorder that gives rise to unregulated erythrocytosis. A one example, a JAK2V617F mutation causes PV.

As used herein, “Montelukast” refers to a member of the leukotriene receptor antagonist (LTRA) category of drugs having formulas including: (R-(E))-1-(((1-(3-(2-(7-Chloro-2-quinolinyl)ethenyl)phenyl)-3-(2-(1-hydroxy-1-methylethyl)phenyl)propyl)thio)methyl)-cyclopropaneacetic acid, and 1-[[[(1R)-1-[3-[(1E)-2-(7-chloro-2-quinolinyl)ethenyl] phenyl]-3-[2-(1-hydroxy-1-methylethyl)phenyl]propyl]sulfanyl]methyl]cyclopropaneacetic acid.

As used herein, “leukotriene receptor antagonist” or “LTRA” or “leukotriene modifier” refers to a compound that inhibits leukotrienes from binding to their receptors.

As used herein, “leukotriene receptor” refers to a receptor activated by bound leukotrienes, typically a G protein-coupled receptor.

As used herein, “leukotriene” generally refers to members of a family of molecules synthesized (derived) from arachidonic acid.

As used herein, “leukotriene inhibitor compound” refers to a leukotriene receptor antagonist and a leukotriene synthesis inhibitor compound.

The term “substitute for” as used herein, refers to the switching the administration of a first compound or drug to a subject for a second compound or drug to the subject.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc.) to a binding partner under conditions such that the binding partner becomes unresponsive to its natural ligands. Inhibitory compounds may include, but are not limited to, nucleic acids, small organic molecules, antibodies, and proteins/peptides.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “test compound” as used herein, refers to any compound or molecule considered a candidate as an inhibitory compound.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

### DETAILED DESCRIPTION OF INVENTION

This invention relates to treating Myeloproliferative neoplasms (MPNs), specifically Polycythemia Vera (PV). In particular, the invention relates to compositions and methods for inhibiting Polycythemia Vera (PV)-initiating cells, e.g. subsets of hematopoietic stem cells that cause PV symptoms. For one example, Montelukast (e.g., Singulair®) may be used for preventing the development of PV or reducing PV symptoms over long periods of time. Further, Montelukast in combination with other compounds, e.g., JAK2 inhibitor compounds, may also find use for treating patients genetically at risk for developing PV and for treating PV patients.

### I. Myeloproliferative Neoplasms (MPNs)

In one embodiment, the present invention contemplates methods of treatments for treating Myeloproliferative neoplasms (MPNs). Thus, in some embodiments, methods of treatments described herein are contemplated for use to provide an additional benefit of reducing at least one symptom of a myeloproliferative neoplasm associated with PV in a patient. Further, in some embodiments, methods of treatments described herein are contemplated for use to provide a benefit of reducing at least one symptom of a myeloproliferative neoplasm in a patient, i.e. for treating patients having other types of myeloproliferative neoplasms.

Myeloproliferative neoplasms (MPNs) refer to a group of hematologic malignancies where there is a physiological disorder in hematopoietic stem cells (HSCs) leading to a myeloproliferative phenotype^{1,2}. MPNs are also described as clonal proliferative disorders where along the pathway between HSCs and progeny cells, a somatic mutation occurs affecting different myeloid lineages associated with MPNs, including but are not limited to chronic myeloid leukemia (CML), polycythemia vera (PV), essential thrombocythemia (ET), and primary myelofibrosis (PMF)³⁻⁶. PV, a common MPN in humans, refers to a clonal stem cell disease characterized by clinical symptoms including erythrocytosis⁷. Thus, along the differentiation-maturation pathway of HPCs, one of the progeny cells had at least one mutation occur that was passed along into a larger progeny cell population. Other symptoms of PV disease may include, but are not limited to, blood leukocytosis, thrombocytosis, and a propensity for disease progression leading to myelofibrosis and/or acute leukemia⁸.

In 2005, four different research groups identified an identical point mutation (e.g., JAK2V617F) in PV patients at an allele encoding JAK2 tyrosine kinase. JAK2V617F allele was found in a majority of patients with PV. JAK2V617F allele was also discovered within in a large proportion of patients with ET and PMF⁹⁻¹². Other mutations in JH2 of JAK1-3 were linked to leukemias such as acute lymphoblastic leukemia and acute myeloid leukemia. Each of these mutations also resulted in constitutive tyrosine kinase activity of JAK2. V617F in JH2 of JAK2 is commonly identified as a mutation in MPN, for >95% of cases of polycythemia vera and ˜50% of cases of essential thrombocythemia and primary myelofibrosis. This mutation was also implicated in non-small-cell lung cancer.

JAK2 is one of at least 4 members of the Janus Kinase (JAK1-4) family of non-receptor tyrosine kinases. Membrane associated JAK2 kinase is involved with transmembrane signaling when associated cell surface type I cytokine receptors bind to extracellular ligands. Examples of JAK2 associated receptors include, but are not limited to, those for growth hormone, erythropoietin (EPO), leptin, interferon-γ and interleukins IL-3 and IL-5, thrombopoietin and granulocyte macrophage colony-stimulating factor (GM-CSF)¹³.

Under physiological conditions, JAK2 is activated when ligands, such as cytokines, bind to type I cytokine receptors, then in turn activated JAK2 transducers this ligand binding signal by phosphorylation of associated downstream signal transducer proteins and activator of transcription (STAT) molecules, leading to activation of JAK2-STAT signaling pathways¹⁴. Therefore, JAK2 is activated through ligand mediated (e.g., cytokine-mediated) receptor dimerization or rearrangement then signals through the JAK-Stat (signal transducer and activators of transcription) pathway. Depending upon the development and differentiation stage of HPCs or progeny cells, JAK-Stat activation may control myeloid cell development, proliferation and survival, as well as initial stages of the immune response.

JAK2 protein kinases comprises at least an N-terminal FERM (e.g., band 4.1, ezrin, radixin, moesin) domain, a Src homology-2 (SH2)-like domain, a pseudokinase domain (JAK homology-2, JH2), and a C-terminal tyrosine kinase domain (JH1). The FERM domain is primarily responsible for the association of JAKs with cytokine receptors. The SH2 kinase domain comprises a pseudokinase domain (JH2), as a phosphotyrosine-binding domain that regulates the catalytic activity of the tyrosine kinase domain (JH1) through controlling the phosphorylation state of JH1. In fact, JH1 is activated via trans-phosphorylation of tandem tyrosines in the activation loop (comprising Tyr1007 and Tyr1008 in JAK2). Hyperactivating mutations in the JH2 region of human JAK2 proteins were found as causative for myeloproliferative neoplasms (MPNs).

Based on structural analysis of JAK2 proteins, the V617F allele has a one amino acid mutation in the expressed protein, where a larger bulky phenylalanine (F) replaces the smaller valine (V) amino acid (i.e. a V to F substitution (V617F mutation) caused by a nucleic acid mutation in the JAK2 gene which upon expression causes the substitution of phenylalanine (F) for valine (V) at position 617 of the Janus kinase (JAK) 2 protein, thus destabilizing the pseudokinase domain (JH2) and a tyrosine kinase domain (JH1) domain of JAK2. Structural and biochemical data indicate that the V617F mutation rigidifies α-helix C in the N lobe of JH2, which then facilitates continual trans-phosphorylation of JH1 instead of maintaining a state of autoinhibition as a flexible helix for phosphorylation of JH1 triggered upon binding of a ligand to an associated receptor. However, other mutations in the JAK2 gene may also cause a gain-of function of the JAK2 enzymatically active protein. Thus, a lack of autoinhibition results in constitutive activation of JAK2 signaling, including constitutive activation of the JAK2-STAT pathway regardless of the presence of bound ligands¹⁰. Constitutive activation of JAK2-STAT pathway in JAK2V617F-positive cells results in overgrowth of the mutant JAK2 containing cells when co-cultured with normal hematopoietic cells¹⁵⁻¹⁷.

### II. Polycythemia Vera

Polycythemia Vera (PV) is a disease in need of more effective and safe treatments, in part due to the need for long term treatment throughout the patient's life. Long term treatment is needed as a prophylactic treatment of patients at risk of developing PV and for delaying or preventing onset of PV symptoms. Further, once PV symptoms arise, then the patient needs lifetime treatments for reducing symptoms. Such effective treatments should additionally have fewer toxic side effects, especially over time, than treatment compounds currently in use.

Therefore, treatments described herein are contemplated to have at least one or more advantages over current treatments including but not limited to: better efficacy, fewer toxic side effects during treatments; fewer toxic side effects accumulated over time, lower effective doses of individual bioactive compounds in combination treatments. Effective doses may be used for numerous intermittent courses of treatments, e.g. as needed; effective doses may be used for consistent treatment over the lifespan of patients at risk of developing PV (as a prophylactic treatment) and/or patients having PV symptoms; may provide “curative” properties, in particular for reducing the bodily population of PV inducing hematopoietic cells.

In particular, patients at risk of developing PV or in the process of developing PV or having PV, may be individually identified as having: increased amounts of arachidonate 5-lipoxygenase (Alox5) and/or overactive Janus kinase 2 activity (e.g., increased amounts of phosphorylated JAK2) in cell samples or biopsy samples comprising hematopoietic cells. Such increased amounts of arachidonate 5-lipoxygenase (Alox5) and phosphorylated Janus kinase 2 may be detected using standard assays, such as biochemical, flow cytometric, cell culture, genetic and immunohistological techniques. Further, a patient at risk of developing PV or in the process of developing PV or having PV, may be identified by a genetic analysis for detecting at least one JAK2V617F allele or other JAK2 mutant allele resulting in increased activation of Janus kinase 2 activity over normal-wildtype, or additional allelic mutations in non JAK2 genes associated with overactive Janus kinase 2. Thus, inhibitor compound treatments may target either Alox5 (expression or synthase activity) or JAK2 activity or both of Alox5 (expression or synthase activity) and JAK2 activity.

Further, methods of treatments described herein may have courses of treatment interspersed over the lifespan of a patient or treatment may be continuous over the lifetime of the patient. Such courses and length of treatment are contemplated to depend upon any one or more non-inclusive factors such as by identifying patients having a JAK2V617F allele prior to onset or at onset of a PV symptom for prophylactic treatment, disease severity at the time of onset of a particular method of treatment, treatments initiated in order to reduce or prevent reoccurrence of PV symptoms, etc. Interspersing courses of treatments may include, but are not limited to, using the same bioactive for each treatment or changing the bioactive drug compound(s) from one course to the next course or when starting a subsequent course of treatment. Continuous treatment may include, but is not limited to, using the same bioactive over time or changing the bioactive drug compound(s) used for treatment during a course of treatment. In some embodiments, a PV treatment is a Cysteinyl leukotriene receptor 1 (CysLT1) antagonist, e.g., montelukast, zafirlukast, and pranlukast, each alone, or in combination with a kinase activity inhibitor compound, e.g., a Janus kinase 2 (JAK2) activity inhibitor compound, a Janus kinase 1/2 (JAK1/2) inhibitor compound (examples provided herein), or a tyrosine kinase activity inhibitor compound (examples provided herein). In one embodiment, said kinase inhibitor compound is not an imidazopyridine compound. In one embodiment, said kinase inhibitor compound is not a heteroaryl kinase inhibitor

In some embodiments, a PV treatment is a leukotriene synthesis inhibitor compound, e.g., zileuton and Hypericum perforatum, each alone, or in combination with a kinase activity inhibitor compound, e.g., a Janus kinase 2 (JAK2) activity inhibitor compound, a Janus kinase 1/2 (JAK1/2) inhibitor compound (examples provided herein), or a tyrosine kinase activity inhibitor compound (examples provided herein). A nonlimiting example of a leukotriene as used herein associated with a drug target, includes but is not limited to a cysteinyl leukotriene (CysLT). CysLT is derived from arachidonic acid through a 5-lipoxygenase (5-LO) synthase pathway. Cysteinyl leukotrienes (Cys-LT; CysLT) includes but are not limited to leukotrienes LTC₄, LTD₄, LTE₄, etc.

Thus, merely for providing a non-limiting example of treating a patient starting with Montelukast alone for one or more courses of treatment, followed by a combination of Montelukast with a Janus kinase 2 activity inhibitor compound for one or more courses of treatment. In other exemplary embodiments, a patient may be treated using Zileuton with a Janus kinase 2 activity inhibitor compound for one or more courses of treatment, optionally followed by additional courses of treatment using Montelukast alone or Montelukast with a Janus kinase 2 activity inhibitor compound. In other embodiments, changing the type of Janus kinase 2 activity inhibitor compound for subsequent courses of treatments is contemplated. These examples are not limiting such that exemplary treatments described herein, may be used as needed, in part based upon one or more factors such as how the patient is responding, whether a patient develops resistance to a particular treatment, or develops adverse side effects, etc.

### III. Arachidonate 5-Lipoxygenase (Alox5)

Alox5 was reported to regulate numerous physiological and pathological progresses, including inflammation and cancer^{18,19}. It has previously been shown that Alox5 is believed to be a genetic effector of JAK2V617F in driving PV. Alox5 is upregulated by JAK2V617F and genetic deletion of Alox5 moderates PV development induced by JAK2V617F in mice.

Meanwhile, Alox5 inhibition reduces the ability of human JAK2V617F-expressing CD34+ cells in colony formation²⁰. These results suggest Alox5 as a candidate therapeutic target for JAK2V617F-induced PV therapy. However, the molecular mechanism by which JAK2V617F-induced PV is suppressed whereby Alox5 inhibition remains unclear.

Previously, arachidonate 5-lipoxygenase (Alox5) was shown as a driver in PV. Inhibition of the Alox5 pathway attenuates PV development in mice induced by JAK2V617F. However, the molecular mechanism underlying the function of Alox5 in PV remains elusive. Leukotrienes are downstream molecules synthesized by Alox5 in leukocytes and play a role in the Alox5 pathway.

### IV. Leukotrienes

Leukotrienes refer to a family of eicosanoid inflammatory mediators synthesized from arachidonic acid (AA) by 5-lipoxygenase (5-LO)²²⁻²⁴. For example, leukotrienes serve as downstream lipid metabolites in an Alox5 pathway involved in inflammation, allergy, as well as in metabolism of some cancer cells²⁵⁻²⁷. Although previous data showed Alox5 as a potential target in the JAK2 signaling pathway for suppressing PV development, its role in relation to leukotrienes was not known in relation to JAK2V617F-induced PV.

A. Type 1 cysteinyl leukotriene receptor (cysLTR1) antagonist.

Montelukast, refers to an FDA-approved anti-human asthma drug, functioning as a type 1 cysteinyl leukotriene receptor (cysLTR1) antagonist that blocks the action of leukotrienes resulting in reduced inflammation response²¹. Other names include but are not limited to: Singulair; Lukotas; Monocast; Monteflo; Montelo-10; Montene; Respaire; Surfair; Telumantes; Ventek; Ventilar; Xalar; Zespira; etc.

As data shows herein, Montelukast inhibits cell growth and induces cell apoptosis in JAK2V617F-cell lines. Further, in vivo studies demonstrated that Montelukast treatment suppresses the development of PV induced by JAK2V617F in mice comparable to the effect of Alox5 inhibition on PV development. The inhibitor compound effect of Montelukast on JAK2V617F-induced PV is contemplated to be dependent on selectively eradicating (or significantly reducing) polycythemia vera causing stem cells while sparing their normal counterparts. Thus, treatment of PV in humans is contemplated using Montelukast. Moreover, it was discovered that Montelukast has a synergetic effect with JAK2 inhibitor compounds on JAK2V617F-cell lines. These findings support Montelukast as a candidate bioactive agent as a leukotriene receptor antagonist synergetic with JAK2 inhibitor compounds in PV treatment.

The following references provide information on Montelukast as a leukotriene antagonist, or as an inhibitor compound of leukotriene synthesis in relation to its potential use for specifically treating patients having polycythemia vera symptoms.

“Montelukast as a compound” downloaded Jul. 10, 2019 (website: pubchem.ncbi.nlm.nih.gov/compound/-Montelukast#section=UN-GHS-Classification), herein incorporated by reference in its entirety. This reference provides a collection of information on Montelukast-Singulair as a leukotriene antagonist; and a member of quinolines, a monocarboxylic acid and an aliphatic sulfide chemical. Information includes chemical structures, numerous related chemical structures and chemical names, and links to drug use and clinical trial information. There is no mention of polycythemia vera nor the term ‘myeloproliferative’.

WO2018204764, Yuting, et al., “Identification And Targeted Modulation Of Gene Signaling Networks.” Publication Date: Aug. 11, 2018, herein incorporated by reference in its entirety. This reference describes numerous cell-signaling antagonists and small molecule drugs, including Montelukast, Montelukast sodium, pranlukast, and zafirlukast, for treating any one of a laundry list of diseases including myeloproliferative disorders and Polycythemia Vera. CysLTR₁, (line 7, page 1275) is listed with a plethora of other receptors as “non-expressed Group 1 of non-expressed genes”, presumably in hepatocytes, however there is no context for why CysLTR1 is listed here. This reference does not explicitly describe using only Montelukast for treating patients having Polycythemia Vera.

EP1958637A1. Wolff, et al., “Pharmaceutical composition for the treatment of IL-8 mediated diseases.” Published Aug. 20, 2008, herein incorporated by reference in its entirety. This reference describes using 1,6-Bis-[3-(3-carboxymethylphenyl)-4-(2-α-D-manno-pyranosyloxy)-phenyl] hexane in combination with leukotriene receptor antagonists and leukotriene synthesis inhibitor compounds such as Montelukast, zafirlukast, and zileuton, for treating Polycythemia Vera, listed as one of many diseases. This reference does not explicitly describe using only Montelukast for treating patients having Polycythemia Vera.

U.S. Pat. No. 9,403,801, Xi et al., “Substituted Heteroaryl Compounds And Methods Of Use.” Publication Date: Feb. 8, 2016, herein incorporated by reference in its entirety. This reference describes combining Montelukast as a leukotriene antagonist, as an inhibitor compound of leukotriene synthesis, with a heteroaryl kinase inhibitor compound for treating a long list of diseases including polycythemia vera. Moreover, a JAK2 mutation (JAK2V617F) is described associated with myeloproliferative disorders such as polycythemia vera, essential thrombocythemia and idiopathic myelofibrosis, etc. There is no explicit mention of administering only Montelukast alone for treating polycythemia vera.

U.S. Patent Application No. 20140206702, Lai, et al., “Imidazopyridine compounds, compositions and methods of use.” Publication Date: Jul. 24, 2014, herein incorporated by reference in its entirety. This reference describes imidazopyridine compounds as inhibitor compounds of TYK2 kinase used in combination with leukotriene modifiers, such as Montelukast, zafirlukast and zileuton, useful for therapy and/or prophylaxis in a patient, for treating diseases including myeloproliferative disorders polycythemia vera, essential thrombocytosis, myelofibrosis or chronic myelogenous leukemia (CML). This reference also states that kinase-activating mutations in JAK2 (e.g., JAK2 V617F) are associated with myeloproliferative disorders (MPDS) in humans. There is no explicit mention of administering Montelukast alone for treating polycythemia vera.

U.S. Pat. No. 8,969,363, Castro, et al., “Heterocyclic compounds for use in the treatment of pi3k-gamma mediated disorders.” Publication Date: Jan. 31, 2013, herein incorporated by reference in its entirety. This reference describes leukotriene receptor antagonists such as Montelukast (Singulair®), and zileuton (Zyflo®), albeit co-administered with compounds capable of selectively inhibiting certain isoform(s) of class I PI3K for treating bone marrow fibrosis associated with a hematologic disorder (e.g., a hematologic disorder including polycythemia vera). This publication also describes treating a human cell population enriched for CD19+ CD34+ B cell progenitors with the compounds listed above. There is no explicit mention of administering Montelukast (Singulair®) alone for treating polycythemia vera.

U.S. Patent Application No. 20160219845, Gaitanaris, et al., “G Protein Coupled Receptors And Uses Thereof.” Publication Date: Apr. 8, 2016, herein incorporated by reference in its entirety. This reference describes using polypeptide antagonists (e.g., peptides and antibodies) and small molecules (e.g., compounds or drugs) for G Protein Coupled Receptors (GPCRs), including antagonists for CysLT₁, where CysLT1 is in a list with numerous other receptors, for treating any one of a laundry list of diseases including myeloproliferative disorders and Polycythemia Vera. However, there are no specific names of therapeutic agents, no less the term Montelukast, for specifically inhibiting a CysLT₁ receptor or for treating myeloproliferative disorders and Polycythemia Vera in particular.

Chen, et al., “Alox5 Blockade Eradicates JAK2V617F-Induced Polycythemia Vera in Mice.” Cancer Research. 77(1):164-174 (2016), herein incorporated by reference in its entirety. This reference describes inhibition of Alox5 function, including treatment with Zileuton, for reducing proliferation of human CD34⁺ cells from Polycythemia Vera (PV) patients. Hematopoietic stem cells (HSCs) harbor JAK2V617F in patients. Zileuton attenuates the development of PV in mice by inhibiting Alox5 function in turn induced by the presence of the JAK2V617F mutation which induces variable PV-like myeloproliferative disease. Alox5 is identified as a candidate therapeutic target for treating refractory myeloproliferative neoplasm where JAK2V617F effects induce PV. JAK2 (JAK2V617F, in a Janus family of non-receptor tyrosine kinases) is found in Philadelphia chromosome-negative myeloproliferative neoplasms (MPN), in approximately 95% of patients with polycythemia vera (PV), 60% of patients with essential thrombocytosis, and 50% of patients with primary myelofibrosis. JAK2V617F-positive cells outpace normal hematopoietic cells as a result of its constitutively active kinase activity and activation of growth factor signaling. JAK2 inhibitor compounds alone, improve symptoms but are not curative. There is no mention of using Montelukast nor an inhibitor compound of a Cysteinyl leukotriene receptor 1 (CysLT₁) (Leukotriene C4 (LTC4)/Leukotriene D4 (LTD4)) receptor for treating Polycythemia Vera.

Yektaei-Karin, et al., “Modulation of leukotriene signaling inhibiting cell growth in chronic myeloid leukemia.” Leuk Lymphoma 58: 1903-1913, 2017, herein incorporated by reference in its entirety. Abstract only. This reference's abstract states that cysteinyl-LT1-receptor-antagonist Montelukast significantly reduced the growth of chronic myeloid leukemia (CML) associated cell lines: K562, KCL22, and KU812 cells, as well as primary CD34⁺ blood cells from two CML patients. There was no mention of Polycythemia Vera.

### V. Montelukast Inhibition of Cell Growth and Apoptosis Induction

Effects of Montelukast on JAK2V617F expressing cell lines were tested. Mouse Ba/F3 and JAK2V617F-expressing Ba/F3 cells, as well as JAK2V617F-expressing human HEL cells treated with Montelukast in various doses for 24 and 48 hours, respectively.

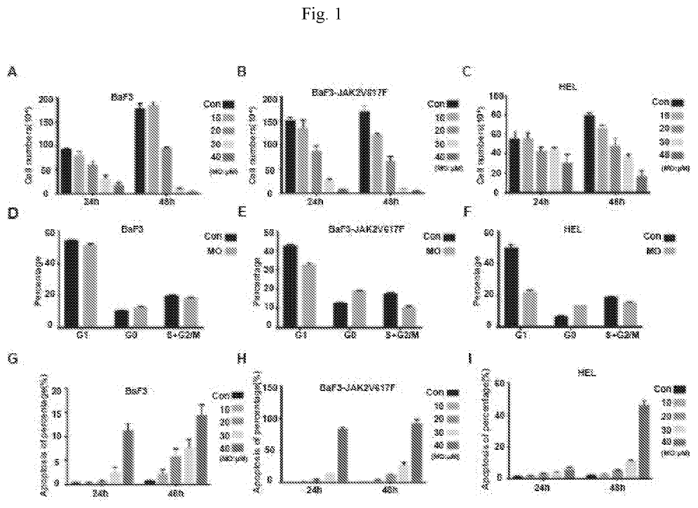

There is dose-dependent growth inhibition observed with Montelukast treatment at both time points (FIGS. 1A, 1B and 1C). To investigate how to inhibit cell growth whereby Montelukast treatment, cell-cycle progression and apoptosis assays were done through flow cytometric (FCM) analysis. Montelukast treatment caused a blockade of cell-cycle progression from G₀ to G₁ phase and G₁ to S+Gr−M phase in both Ba/F3-JAK2V617F and HEL cells (FIGS. 1E and 1F).

In contrast, Montelukast had no significant block effect on parental Ba/F3 cells that do not express JAK2V617F (FIG. 1D). In addition, Montelukast treatment induced a more dramatic apoptosis in Ba/F3-JAK2V617F and HEL cells compared with Ba/F3 cells (FIGS. 1G, 1H and 1I). Together, these results indicate that Montelukast inhibits cell growth and induces cell apoptosis in a JAK2V617F-dependent way.

### VI. Montelukast Reduction OF Polycythemia Vera Development

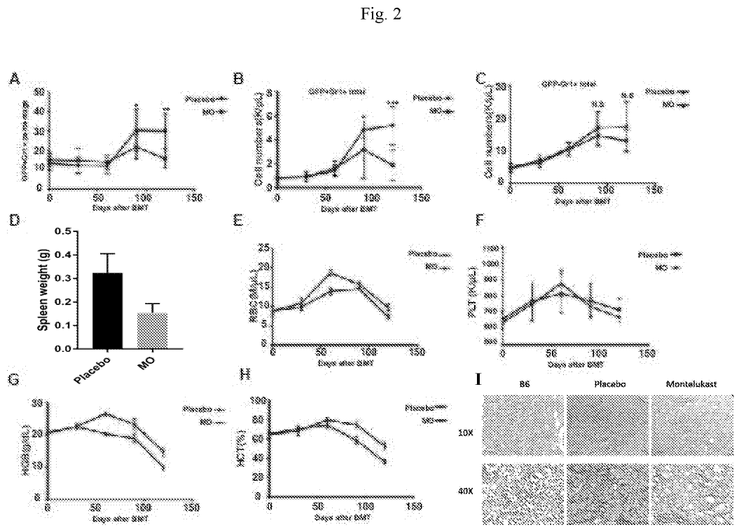

To further evaluate the effect of Montelukast on PV development, PV was induced in mice and then PV mice were treated with Montelukast. The bone marrow cells from donor mice, pretreated with 5-fluorouracil (5-FU), were infected with retrovirus containing pMSCV-JAK2V617F-GFP under conditions for the induction of PV as described previously, followed by transplantation of the transduced cells into C57BL/6J recipient mice. Then the mice were treated with placebo or Montelukast after the development of PV disease based on FCM data. In peripheral blood (PB) of PV mice, both GFP+Gr−1+PV cell number and percentage increased more slowly with the treatment of Montelukast compared with placebo-treated group from 3rd month post treatment (FIGS. 2A and 2B). In contrast, GFP−Gr−1+ cell number didn't show significant difference between two groups (FIG. 2C), suggesting that Montelukast specifically suppressed the PV cells without sparing normal cells. The effect of Montelukast on PV development is consistent with PB as evidenced by less PV cell infiltration to the spleens compared to the placebo-treated group (FIG. 2D). Further, the effect of Montelukast on erythroid cells in PV development was evaluated. Montelukast did not significantly affect the level of RBC and PLT (FIGS. 2E and 2F) but had an inhibitor compound effect on HGB and HCT (FIGS. 2G and 2H). Surprisingly, Montelukast treatment efficiently relieved the fibrosis in bone marrow of PV mice (FIG. 2I). Taken together, these results demonstrate that Montelukast suppresses PV development induced by JAK2V617F in mice.

### VII. Montelukast Impairment of Polycythemia Vera Stem Cell Function

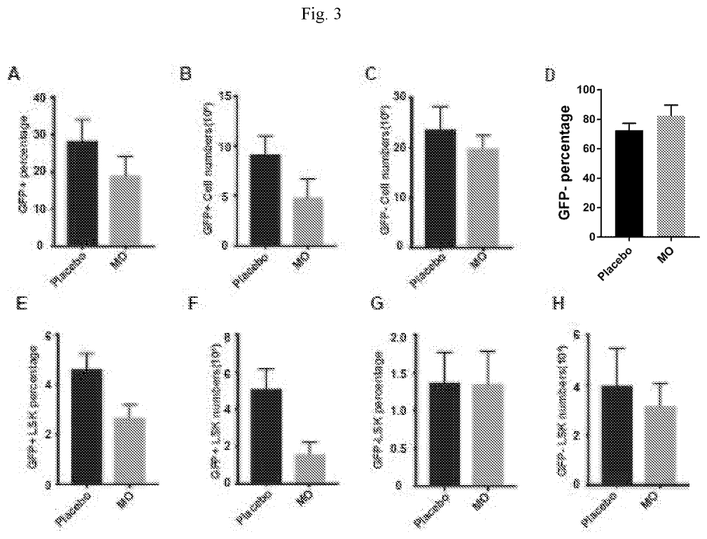

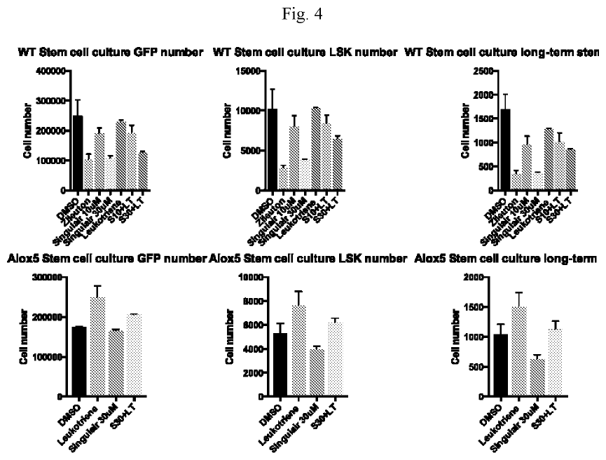

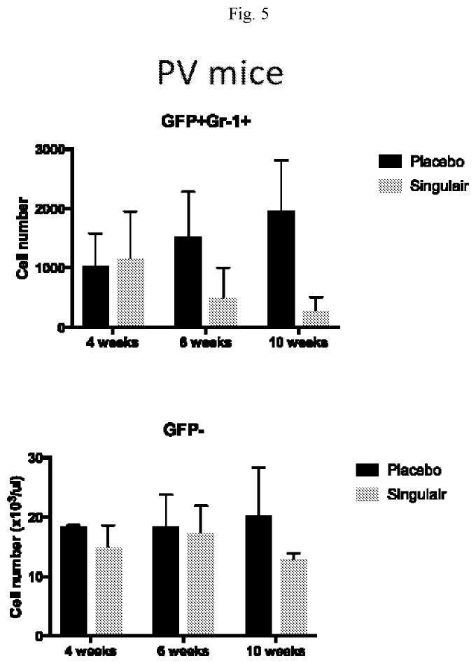

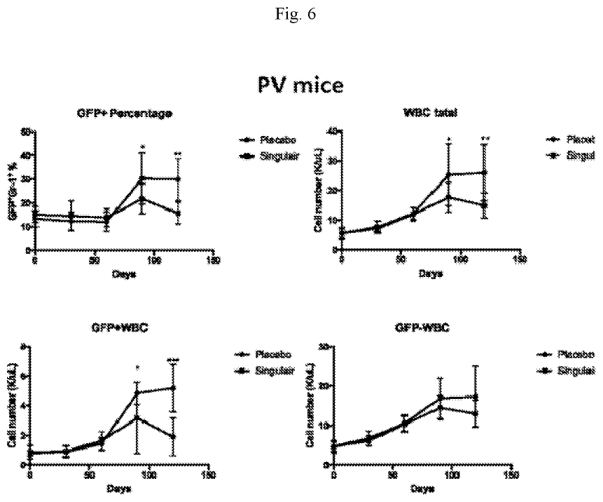

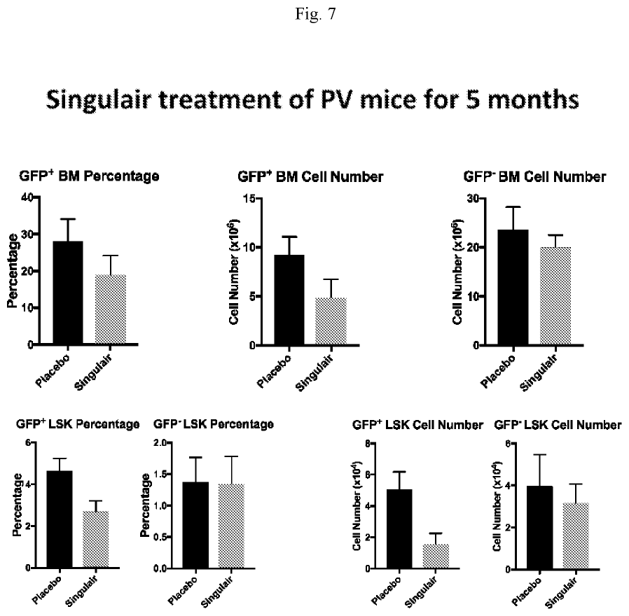

It was contemplated that Montelukast might moderate PV development through inhibiting PSC function. To test this hypothesis, bone marrow cells (BM) were collected for FCM analysis after 4 months treatment. FCM analysis showed that Montelukast treatment caused a dramatic decrease in both GFP+ PV cell number and percentage in BM (FIGS. 3A and 3B). In line with the total BM, both the percentage and number of PSCs (GFP+Lin−c−Kit+Sca−1+) dropped to a much lower level with the treatment of Montelukast compared to placebo-treated group (FIGS. 3E and 3F). Conversely, both GFP− BM cell number and GFP-Lin-c-Kit+Sca−1+ did not show a noteworthy difference between Montelukast-treated group and placebo-treated group (FIGS. 3C, 3D, 3G, and 3H). These data verified the hypothesis that Montelukast inhibits PV development in mice through selectively eradicating PSCs in BM while sparing normal stem cells.

### VIII. Therapeutic Applications

Studies showed that transduction of JAK2V617F in growth factor-dependent Ba/F3 cell line facilitates it to survive in a growth factor-independent way¹⁰. Meanwhile, mice transplanted with JAK2V617F-infected bone marrow cells develop a PV-like disease^{10,16}. These studies indicate JAK2V617F mutation as a genetic leading cause that constitutively activates JAK2 kinase and the downstream signaling pathway in PV development. Hence, JAK2V617F or the downstream signaling molecules could be potential candidate targets for PV therapy. A previous study showed that Alox5 expression is upregulated by JAK2V617F and deletion of Alox5 mitigates PV development in mice, suggesting Alox5 as a possible JAK2V617F downstream target for PV treatment²⁰. Nonetheless, it is not clear how Alox5 function in JAK2 pathway that is responsible for PV development.

Leukotrienes, a family of lipid mediators synthesized by Alox5, are downstream metabolites in Alo5 pathway that was reported to play a role in immune response, inflammatory disorders, and cancer metabolism²⁴⁻²⁸.

The data presented herein suggests that JAK2V617F-induced PV showed an effective response to Montelukast treatment in mice through preventing leukotrienes from binding to their receptors. Montelukast, an antagonist against the leukotriene receptors, blocks the function of leukotrienes and does not affect the enzyme activity of 5-LO and the expressional level of Alox5. Thus, data shown herein indicates that leukotrienes in the Alox5 pathway are involved with JAK2V617F-induced PV in mice.

The identification of JAK2V617F mutation deepens an understanding of the molecular pathogenesis of MPNs. JAK2V617F alone is sufficient to induce MPNs phenotype in vitro and in vivo models¹⁰, which confers JAK2V617F as a driver mutation in MPNs and renders it to be a logical target for therapeutic intervention. Hence, subsequent studies were focused on the inhibition of JAK kinase activity caused by JAK2V617F in MPNs treatment. As one example, Ruxolitinib, a small-molecule inhibitor compound of JAK1/2, was approved by the United States Food and Drug Administration (FDA) for treatment of patients with myelofibrosis, and then for treatment of patients with postpolycythemia vera myelofibrosis and polycythemia vera (e.g., Ruxolitinib of 10 mg twice daily) who had an inadequate response to or are intolerant of hydroxyurea²⁹.

Despite success in targeted therapy for MPNs using JAK2 inhibitor compounds, they do not induce an optimal molecular remission due to the inability to eliminate MPN initiating stem cells¹⁵. MPNs stem cells (MSCs) are defined as a rare hematopoietic population harboring an acquired JAK2V617F somatic mutation that is responsible for MPN initiation, disease progression and drug resistance³⁰. Similar to Leukemic stem cells (LSCs), MSCs are also less sensitive or even resistant to the treatment of tyrosine kinase inhibitor compounds¹⁵. Increasing evidence has shown that MSCs are involved in MPN and targeting MSCs may offer a curable treatment option for MPNs patients³⁰. As shown herein, data demonstrated that Montelukast has a dramatic inhibitor compound effect on PSCs in JAK2V617F-induced PV mice. This result suggests that leukotrienes are present for PSCs function in JAK2V617F-induced PV and block of leukotrienes using antagonists ameliorates PV development in mice.

JAK2 inhibitor compounds cannot eliminate JAK2V617F initiating PSCs, which suggests that PSCs function independently of the kinase activity of JAK2. Similarly, a previous study relating to Alox5 function showed that Ruxolitinib blocks JAK2 phosphorylation but does not affect 5-LO expression, suggesting that Alox5 pathway functions in JAK2V617F initiating PSCs independently of its kinase activity as well²⁰. This evidence supports a point of view that the inhibition of Alox5 pathway through blocking leukotrienes binding impairs the function of JAK2V617F initiating PSCs, which is working in a different way from JAK2 inhibitor compounds that blocks the kinase activity in the treatment of JAK2V617F-induced PV. Notably, as shown herein, Montelukast manifested a preferential inhibitor compound effect on PSCs to their normal counterpart hematopoietic stem cells (HSCs), indicating that leukotrienes are functionally more involved with PSCs compared to HSCs.

It is generally accepted that eradication of cancer stem cells could result in the therapeutic cures for cancers, but a major challenge is how to specifically target cancer stem cells while sparing normal stem cell counterparts. Recent advances in identification of genes required for survival of cancer stem cells as potential drug targets may be used for developing novel therapies aimed to eliminate cancer stem cells. However, a potential problem of this strategy is that potential target genes are usually expressed in normal stem cells as well, which renders these cells to be targeted simultaneously leading to severe side effects. For example, JAK2 plays a role in regulating the function of MSCs. However, normal HSC function is evidenced by studies showing that genetic deletion of JAK2 in mice leads to severe defects in normal HSCs function^{31,32}.

Hence, success of an anti-stem cell therapy may rely upon on inhibition of genes that are more functional in the maintenance of cancer stem cells than their normal counterparts. Some studies have shown that although certain genes play roles in both cancer and normal stem cells, they are functionally more involved with cancer than for normal stem cells^{33,34}. In this situation, the difference in the degree of dependence on the same genes for survival between cancer and normal stem cells provides a therapeutic window for more specifically targeting therapeutics at cancer stem cells³⁵.

This study further provides supporting evidence for testing and using an effective therapeutic strategy for PV patients, including preventative therapy, through specific targeting of PSCs.

### IX. Human Polycythemia Vera Therapy

Normally, a human body regulates the number of at least three types of blood cells produced by the bone marrow, including red blood cells, white blood cells and platelets. But in polycythemia vera, the bone marrow makes too many of some of these blood cells. Signs and symptoms of PV may be similar to those of other myeloproliferative neoplasms while many people with polycythemia vera do not experience noticeable signs or symptoms. Some people with PV might develop vague symptoms such as headache, dizziness, fatigue and blurred vision. However, other people with PV may have more-specific symptoms of polycythemia vera including but not limited to: itchiness, especially after a warm bath or shower; numbness; tingling; burning; or weakness in hands; feet; arms or legs; a feeling of fullness soon after eating and bloating or pain in the left upper abdomen due to an enlarged spleen; unusual bleeding; such as a nosebleed or bleeding gums; painful swelling of one joint, often the big toe; and shortness of breath and difficulty breathing when lying down. Risk factors for people who may develop PV, include but are not limited to genetic factors, for examples, having at least one JAK2V617F allele; having an allele that results in the development of PV, and the like, particularly located in and expressed by subsets of hematopoietic stem cells that cause or are associated with inducing human PV symptoms. Both children and adults may be at risk for, and develop, PV symptoms. Therefore, treatment may extend from a few weeks up to the lifetime of a patient.

In one embodiment, a leukotriene receptor antagonist is contemplated for use in treating PV patients. In one embodiment, a leukotriene receptor antagonist is contemplated for use in slowing the progress of PV in patients. In one embodiment, a leukotriene receptor antagonist is contemplated for use in slowing the age of onset of or preventing the development of PV symptoms in patients at risk of developing PV, e.g., having at least one PV susceptibility allele, e.g., JAK2V617F.

In one embodiment, a leukotriene receptor antagonist, specifically Montelukast sodium, may be administered in doses ranging from 1, 2, 3, 4, 5, 10 mgs. A dose may be administered as a single daily dose or daily as multiple doses, e.g., in the morning, in the evening, and/or during the day. A dose may be administered with or without food. A dosage may be based upon one or more variables such as age and medical condition. Administration may be as oral granules, a chewable tablet and a tablet for swallowing, with or without food. As one non-limiting example, a 10 mg tablet may be administered once a day for 8 weeks, up to one year, or up to the lifetime of a patient. Administration may be continuous, or stop and start, depending upon the medical condition of the patient-severity of PV symptoms. In some embodiments, PV patients with asthma may follow instructions for taking Singular as an asthma treatment with the added benefit of reducing at least one or more PV symptom, or the severity of at least one PV symptom, or for the added benefit of preventing the onset of a PV symptom.

In preferred embodiments, a therapeutic treatment is contemplated for both short term and long term therapy of human PV patients. In some embodiments, a preventative treatment may last the reminder of the life of a patient at risk of developing PV. A PV patient may have one or more PV symptoms, e.g. fatigue, etc. A PV patient may be identified as having a JAK2V617F allele. A patient at risk of having at least one symptom of PV may be identified as having a JAK2V617F allele.

In one embodiment, a leukotriene receptor antagonist, e.g., Montelukast, Zileuton, etc., may be co-administered with a JAK2 inhibitor compound to a patient suspected of or known to have PV.

Thus, in some embodiments, Montelukast may be co-administered with a JAK2 inhibitor compound to a patient suspected of or known to have PV. JAK2 inhibitor compounds that were determined, or contemplated to be determined, to be safe for use in humans, including but not limited to Fedratinib (SAR302503; trade name Inrebic), ruxolitinib, SAR317461, Gandotinib (LY-2784544) and the like, may be used for treatment. In some embodiments, the JAK2 inhibitor compound for co-administration with Montelukast, may be a JAK2 inhibitor compound known for use to treat any one or more of myeloproliferative neoplasms, myeloproliferative disorders, etc. In some embodiments, co-administration of Montelukast and a JAK2 inhibitor compound is contemplated to provide additional benefits, over Montelukast alone, for treating a larger range of myeloproliferative disease symptoms.

Thus, in some embodiments, Zileuton may be co-administered with a JAK2 inhibitor compound, as described herein, to a patient suspected of or known to have PV. One contemplated total daily dose is 2400 mg, e.g., as 600 mg every 6 hours, two 600 milligram (mg) tablets two times a day, one 1200 mg tablet, two times a day, and the like. Another contemplated dose a daily dose that is a dose that shows efficacy that is less than a total daily dose is 2400 mg. In preferred embodiments, a daily dose of Zileuton is less than 2400 mg. Zileuton may be co-administered as an extended release formulation, i.e., as extended-release tablets, an immediate release formulation, etc. In some embodiments, it is contemplated that by using Zileuton co-administered with a JAK2 inhibitor compound reduces at least one undesirable side effect of using Zileuton alone.

In yet other embodiments, Montelukast may be co-administered with a tyrosine kinase inhibitor compound for reducing the severity of at least one or more PV symptoms. Examples of a tyrosine kinase inhibitor compound include but are not limited to a Bcr-Abl tyrosine kinase inhibitor compound, e.g., imatinib; AMN107; AEE788, a multiple target tyrosine kinase inhibitor compound for reducing phosphorylation of the tyrosine kinases of epidermal growth factor receptor (EGFR), human epidermal growth factor receptor 2 (HER2), and vascular endothelial growth factor receptor 2 (VEGF2), etc. In some embodiments, co-administration is with a treatment for targeting one or more proteins that degrade the target PV protein.

In some embodiments, co-administration, e.g., as a combination treatment, is over the first 3 months of initiating a treatment, followed in additional months by administration of Montelukast alone. Thus, when starting and stopping treatments, co-administration may be used in the first approximately 3 months of treatment, each time a course of treatment begins.

Contemplated advantages of using a leukotriene receptor antagonist, e.g. Montelukast, with or without co-administration of a JAK2 inhibitor compound or tyrosine kinase inhibitor compound, or includes but is not limited to reduced side effects; reduced toxicity; thus avoiding adverse side effects when using anti-cancer treatments, or some types of inhibitor compounds causing undesirable side effects. Contemplated advantages of using a leukotriene receptor antagonist, e.g. Zileuton, with or without co-administration of a JAK2 inhibitor compound or tyrosine kinase inhibitor compound, or includes but is not limited to reduced side effects; reduced toxicity; thus avoiding adverse side effects when using anti-cancer treatments, etc., causing undesirable side effects. Merely for example, in some embodiments, use of treatments described herein are contemplated to have additional benefits over other treatments for patients at risk of or having PV including but are not limited to the reduction of nausea, upset stomach, diarrhea, trouble sleeping, cold symptoms (stuffy nose, sinus pain, sneezing, sore throat), headache, weakness, muscle pain, etc.

### EXPERIMENTAL

The following examples are offered to illustrate various embodiments of the invention, but should not be viewed as limiting the scope of the invention.

### Example I

### Montelukast

One example of a structure of Montelukast sodium:

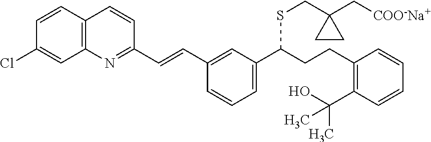

### Example II

### Cell Lines and Mice

A mouse Ba/F3 cell line transduced with pMSCV-IRES-GFP was used as a control. Mouse JAK2V617F-GFP-expressing Ba/F3 cell line was kindly provided by Dr. Ross Levine (Human Oncology and Pathogenesis Program, Memorial Sloan-Kettering Cancer Center, New York, NY). Human JAK2V617F-expressing HEL cell line was purchased from ATCC. Cell lines were cultured in RPMI-1640 medium containing 10% FCS and 50 μmol/L 2-mercaptoethanol. Ba/F3 cell line was cultured in the medium mentioned above with 10 ng/ml IL3. C57BL/6J mice were obtained from The Jackson Laboratory.

### Example III

### Retroviral Bone Marrow Transplantation Model of PV

The pMSCV-JAK2V617F-GFP construct was kindly provided by Dr. Richard Van Etten (Tufts-New England Medical Center). The JAK2V617F retrovirus was produced by co-transfection of 293T cells with pMSCV-JAK2V617F-GFP and ecotropic packaging construct.

PV model was induced in mice as follows: Bone marrow cells from 5-FU-treated (200 mg/kg) donor mice were transduced twice with JAK2V617F retrovirus in the presence of IL-3, IL-6, and stem cell factor (SCF). The recipient mice received 1,100 cGy lethal gamma irradiation (given by 2 split 550-cGy doses), followed by 2.0×10⁶ cells transplantation via tail vein injection.

### Example IV

### Flow Cytometric Analysis of Hematopoietic Cells

Hematopoietic cells were collected from peripheral blood and bone marrow of the PV mice, and red blood cells (RBCs) were eliminated using RBC lysis buffer (pH 7.4). Then the cells were stained with fluorescence-labeled antibodies (Gr−1 for neutrophils, c-kit and Sca−1 for stem cells) and subjected to FACS analysis. These antibodies were purchased from eBioscience Biotechnology. Data were acquired on a FACS Calibur flow cytometric analyzer (Becton Dickinson).

### Example V

### Drug Treatment

PV mice with confirmed disease based on FACS data were randomly divided into the placebo or drug treatment groups' 1-month post bone marrow transplantation (BMT). Montelukast (Camber Pharmaceuticals) was dissolved in water and given orally in a volume of 0.3 mL by gavage (150 mg/kg, once a day) starting from 1 month after BMT. Water was used as placebo.

### Example VI

### Cell Cycle And Apoptosis Assays

Equal number of cells were plated in 24-well plate and treated with various doses (10-40 μM) of Montelukast (Selleckchem) for 24 h and 48 h. Cell numbers were counted in duplicates by trypan blue (Sigma-Aldrich) exclusion at 24 h and 48 h, respectively. FACS analysis was applied for cell cycle test using Hoechst and Pyronin (Sigma-Aldrich) staining, and for apoptosis assay staining with Annexin V and 7-AAD (eBiosciences) according to the manufacturer's instructions. Results are expressed as the mean±SD.

### Example VII

### Western Blot Analysis

Antibodies against p-AKT, AKT, p-PI3K, PI3K, p-MAPK and MAPK were purchased from Cell Signaling Technology. Antibodies against β-catenin and β-actin were purchased from Santa Cruz Biotechnology. Protein lysates were prepared by lysing cells in RIPA buffer and Western blotting was performed for expression of the proteins as mentioned above.

### Example VIII

### Statistical Analysis

Statistical analysis was performed by using the Student t test (GraphPad Prism, *P<0.05; **P<0.01; ***, P<0.001).

### REFERENCES

All publications and patents mentioned in the above specification are herein incorporated by reference. Various modifications and variations of the described methods and system of the invention will be apparent to those skilled in the art without departing from the scope and spirit of the invention. Although the invention was described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, microscopy, genetics, statistics or related fields are intended to be within the scope of the following claims.
1 Dameshek, W. Some speculations on the myeloproliferative syndromes. Blood 6, 372-375 (1951).
2 Tefferi, A. & Vainchenker, W. Myeloproliferative neoplasms: molecular pathophysiology, essential clinical understanding, and treatment strategies. J Clin Oncol 29, 573-582, doi:10.1200/JCO.2010.29.8711 (2011).
3 Fialkow, P. J., Faguet, G. B., Jacobson, R. J., Vaidya, K. & Murphy, S. Evidence that essential thrombocythemia is a clonal disorder with origin in a multipotent stem cell. Blood 58, 916-919 (1981).
4 Yumori, Y., Sugiyama, H., Takahashi, T., Haebara, H. & Hoshino, T. [An autopsy case of acute myelodysplasia with myelofibrosis—cytogenetically proved evidence of a clonal disorder with origin in a multipotent stem cell]. Rinsho Ketsueki 27, 519-525 (1986).
5 Prchal, J. F. & Axelrad, A. A. Letter: Bone-marrow responses in polycythemia vera. N Engl J Med 290, 1382, doi:10.1056/nejm197406132902419 (1974).
6 Arber, D. A. et al. The 2016 revision to the World Health Organization classification of myeloid neoplasms and acute leukemia. Blood 127, 2391-2405, doi:10.1182/blood-2016-03-643544 (2016).
7 Adamson, J. W., Fialkow, P. J., Murphy, S., Prchal, J. F. & Steinmann, L. Polycythemia vera: stem-cell and probable clonal origin of the disease. N Engl J Med 295, 913-916, doi:10.1056/NEJM197610212951702 (1976).
8 Cashman, J. D., Eaves, C. J. & Eaves, A. C. Unregulated proliferation of primitive neoplastic progenitor cells in long-term polycythemia vera marrow cultures. J Clin Invest 81, 87-91, doi:10.1172/JCI113315 (1988).
9 Baxter, E. J. et al. Acquired mutation of the tyrosine kinase JAK2 in human myeloproliferative disorders. Lancet 365, 1054-1061, doi:10.1016/S0140-6736(05)71142-9 (2005).
10 James, C. et al. A unique clonal JAK2 mutation leading to constitutive signalling causes polycythaemia vera. Nature 434, 1144-1148, doi:10.1038/nature03546 (2005).
11 Kralovics, R. et al. A gain-of-function mutation of JAK2 in myeloproliferative disorders. N Engl J Med 352, 1779-1790, doi:10.1056/NEJMoa051113 (2005).
12 Levine, R. L. et al. Activating mutation in the tyrosine kinase JAK2 in polycythemia vera, essential thrombocythemia, and myeloid metaplasia with myelofibrosis. Cancer Cell 7, 387-397, doi:10.1016/j.ccr.2005.03.023 (2005).
13 Parganas, E. et al. JAK2 is essential for signaling through a variety of cytokine receptors. Cell 93, 385-395, doi:10.1016/s0092-8674(00)81167-8 (1998).
14 Lu, X. et al. Expression of a homodimeric type I cytokine receptor is required for JAK2V617F-mediated transformation. Proc Natl Acad Sci USA 102, 18962-18967, doi:10.1073/pnas.0509714102 (2005).
15 Mullally, A. et al. Physiological JAK2V617F expression causes a lethal myeloproliferative neoplasm with differential effects on hematopoietic stem and progenitor cells. Cancer Cell 17, 584-596, doi:10.1016/j.ccr.2010.05.015 (2010).
16 Wernig, G. et al. Expression of JAK2V617F causes a polycythemia vera-like disease with associated myelofibrosis in a murine bone marrow transplant model. Blood 107, 4274-4281, doi:10.1182/blood-2005-12-4824 (2006).
17 Jamieson, C. H. et al. The JAK2 V617F mutation occurs in hematopoietic stem cells in polycythemia vera and predisposes toward erythroid differentiation. Proc Natl Acad Sci USA 103, 6224-6229, doi:10.1073/pnas.0601462103 (2006).
18 Chen, X. S., Sheller, J. R., Johnson, E. N. & Funk, C. D. Role of leukotrienes revealed by targeted disruption of the 5-lipoxygenase gene. Nature 372, 179-182, doi:10.1038/372179a0 (1994).
19 Catalano, A., Rodilossi, S., Caprari, P., Coppola, V. & Procopio, A. 5-Lipoxygenase regulates senescence-like growth arrest by promoting ROS-dependent p53 activation. EMBO J 24, 170-179, doi:10.1038/sj.emboj.7600502 (2005).
20 Chen, Y. et al. Alox5 Blockade Eradicates JAK2V617F-Induced Polycythemia Vera in Mice. Cancer Res 77, 164-174, doi:10.1158/0008-5472.CAN-15-2933 (2017).
21 Markham, A. & Faulds, D. Montelukast. Drugs 56, 251-256; discussion 257, doi:10.2165/00003495-199856020-00010 (1998).
22 Arai, Y. et al. Synthesis and 5-lipoxygenase inhibitor compoundy activities of eicosanoid compounds. J Med Chem 26, 72-78, doi:10.1021/jm00355a015 (1983).
23 Weller, P. F. et al. Generation and metabolism of 5-lipoxygenase pathway leukotrienes by human eosinophils: predominant production of leukotriene C4. Proc Natl Acad Sci USA 80, 7626-7630, doi:10.1073/pnas.80.24.7626 (1983).
24 Stjernschantz, J. The leukotrienes. Med Biol 62, 215-230 (1984).
25 Vincent, C. et al. 5-LOX, 12-LOX and 15-LOX in immature forms of human leukemic blasts. Leuk Res 32, 1756-1762, doi:10.1016/j.leukres.2008.05.005 (2008).
26 Runarsson, G. et al. The expression of cytosolic phospholipase A2 and biosynthesis of leukotriene B4 in acute myeloid leukemia cells. Eur J Haematol 79, 468-476, doi:10.1111/j.1600-0609.2007.00967.x (2007).
27 Romano, M. & Claria, J. Cyclooxygenase-2 and 5-lipoxygenase converging functions on cell proliferation and tumor angiogenesis: implications for cancer therapy. FASEB J 17, 1986-1995, doi:10.1096/fj.03-0053rev (2003).
28 Sharma, J. N. & Mohammed, L. A. The role of leukotrienes in the pathophysiology of inflammatory disorders: is there a case for revisiting leukotrienes as therapeutic targets?Inflammopharmacology 14, 10-16, doi:10.1007/s10787-006-1496-6 (2006).
29 Mascarenhas, J. & Hoffman, R. Ruxolitinib: the first FDA approved therapy for the treatment of myelofibrosis. Clin Cancer Res 18, 3008-3014, doi:10.1158/1078-0432.CCR-11-3145 (2012).
30 Mead, A. J. & Mullally, A. Myeloproliferative neoplasm stem cells. Blood 129, 1607-1616, doi:10.1182/blood-2016-10-696005 (2017).
31 Grisouard, J., Hao-Shen, H., Dirnhofer, S., Wagner, K. U. & Skoda, R. C. Selective deletion of JAK2 in adult mouse hematopoietic cells leads to lethal anemia and thrombocytopenia. Haematologica 99, e52-54, doi:10.3324/haematol.2013.100016 (2014).
32 Akada, H. et al. Critical role of JAK2 in the maintenance and function of adult hematopoietic stem cells. Stem Cells 32, 1878-1889, doi:10.1002/stem.1711 (2014).
33 Zhao, C. et al. Hedgehog signalling is essential for maintenance of cancer stem cells in myeloid leukaemia. Nature 458, 776-779, doi:10.1038/nature07737 (2009).
34 Dierks, C. et al. Expansion of Bcr-Abl-positive leukemic stem cells is dependent on Hedgehog pathway activation. Cancer Cell 14, 238-249, doi:10.1016/j.ccr.2008.08.003 (2008).
Chen, Y. & Li, S. Omacetaxine mepesuccinate in the treatment of intractable chronic myeloid leukemia. Onco Targets Ther 7, 177-186, doi:10.2147/OTT.S41786 (2014).

I claim:

1. A method of treatment, comprising:
a) providing;
i) a patient having Polycythemia Vera or is at risk of having Polycythemia Vera; and
ii) a therapeutic composition comprising an active component consisting essentially of Montelukast; and
b) administering said therapeutic composition to said patient wherein development of said Polycythemia Vera is inhibited development in said patient.

2. The method of claim 1, wherein said Montelukast is selected from the group consisting of Montelukast sodium; Montelukast nitrile; and [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid, monosodium salt).

3. The method of claim 1, wherein said patient comprises hematopoietic stem cells (HSCs) having a JAK2V617F mutation.

4. The method of claim 1, wherein the method further comprises isolating a first sample comprising a first plurality of hematopoietic stem cells from said patient, wherein a percentage of said first plurality of hematopoietic stem cells have over-active Janus kinase 2 (JAK2).

5. The method of claim 4, wherein the method further comprises isolating a second sample comprising a second plurality of hematopoietic stem cells from said patient, wherein a lower percentage of said second plurality of hematopoietic stem cells have over-active Janus kinase 2 (JAK2) as compared to said first plurality of hematopoietic stem cells.

6. The method of claim 4, wherein said first plurality of hematopoietic stem cells further comprise a percentage hematopoietic stem cells that are over-expressing arachidonate 5-lipoxygenase (Alox5).

7. The method of claim 5, wherein said second plurality of hematopoietic stem cells further comprise a lower percentage of said hematopoietic stem cells that are overexpressing arachidonate 5-lipoxygenase (Alox5) than said first plurality of hematopoietic stem cells.

8. The method of claim 1, wherein said patient is human.

9. A method of treatment, comprising:
a) providing;
i) a patient having, or is at risk of having, Polycythemia Vera; and
ii) a therapeutic composition comprising an active component consisting essentially of a combination of montelukast sodium and a JAK2 inhibitor compound; and
b) administering said therapeutic composition to said patient wherein development of said Polycythemia Vera is inhibited.

10. The method of claim 9, wherein said JAK2 inhibitor compound is ruxolitinib.

11. The method of claim 9, wherein said JAK2 inhibitor compound is selected from the group consisting of redratinib, SAR317461, gandotinib, CEP-701

12. A method of treatment, comprising:
a) providing;
i) a patient having, or is at risk of having, Polycythemia Vera; and
ii) a therapeutic composition comprising an active component consisting essentially of a combination of montelukast sodium and a tyrosine kinase inhibitor compound; and
b) administering said therapeutic composition to said patient wherein the development of said Polycythemia Vera is inhibited.

13. The method of claim 12, wherein said tyrosine kinase inhibitor compound is selected from the group consisting of a Bcr-Abl tyrosine kinase inhibitor compound, AMN107; and AEE788.

14. A method of treatment, comprising:
a) providing;
i) a population of CD34+ cells isolated from a patient having, or is at risk of having, Polycythemia Vera; and
ii) a therapeutic composition comprising an active components consisting essentially of a combination of Montelukast and a kinase inhibitor compound; and
b) culturing said CD34+ cells wherein a growing population of said cells is created; and
c) administering said therapeutic composition to said growing population of CD34+ cells, such that cell growth of said growing population of CD34+ is reduced.

15. The method of claim 14, wherein said kinase inhibitor compound is a JAK2 inhibitor compound.

16. The method of claim 14, wherein said kinase inhibitor compound is a tyrosine kinase inhibitor compound.

17. The method of claim 14, wherein said method further comprises isolating first sample from said growing population of CD34+ cells, such that a percentage of said CD34+ cells have over-active Janus kinase 2 (JAK2).

18. The method of claim 14, wherein said method further comprises isolating a second sample from said growing population CD34+ cells, wherein a lower percentage of said CD34+ cells have over-active Janus kinase 2 (JAK2) than said first sample.

19. The method of claim 17, wherein said first sample further comprising a percentage of CD34+ cells that are over-expressing arachidonate 5-lipoxygenase (Alox5).

20. The method of claim 18, wherein said second sample further comprises a lower percentage of said CD34+ cells overexpressing arachidonate 5-lipoxygenase (Alox5) than said first sample.

21. The method of claim 14, wherein said method further comprises treating said patient with said reduced growth CD34+ cell population wherein development of, or a symptom of, Polycythemia Vera is reduced.
